(19) United States
(12) Patent Application Publication
(10) Pub. No.: US 2016/0278989 A1
(43) Pub. Date: Sep. 29, 2016

(54) SURGICAL GAUZE

(71) Applicant: B-RES DI BRAGA ING. GIUSEPPE E C. SAS, Borgosatollo (BS) (IT)

(72) Inventor: Carlo BRAGA, Borgosatollo (BS) (IT)

(73) Assignee: B-RES DI BRAGA ING. GIUSEPPE E C. SAS, Borgosatollo (BS) (IT)

(21) Appl. No.: 15/037,760

(22) PCT Filed: Nov. 19, 2014
(86) PCT No.: PCT/EP2014/075039

§ 371 Date: May 19, 2016

(30) Foreign Application Priority Data
Nov. 22, 2013 (IT) BS2013A000173

(51) Int. Cl. A61F 13/36

(52) U.S. Cl. CPC A61F 13/36

(57) ABSTRACT

A surgical gauze is described, comprising a first layer of absorbent fabric, a second layer of waterproof fabric and a third layer of absorbent fabric. Said second layer is interposed between said first layer and third layer. Said first absorbent layer is on top of the second waterproof layer and is removable from a surgical site to combine the haemostatic, landmark surgery and counting function.

The present invention relates to a surgical device (such as a gauze, swab, sponge) adapted to absorb body fluids, and at the same time be an area of landmark surgery for the operator for facilitating the operation because it is easily distinguishable to the human eye and allows identifying and locating the target of surgery more easily.

### BACKGROUND OF THE INVENTION

Moreover, it allows the operator to maximize the functionality of his work by controlling the hemorrhage and intervene with a significantly more focused view of the surgical site.

This also obviates the possible x-ray examination, if we consider the current surgical gauzes provided with rx detectable material, if the gauze counting system highlights a discrepancy in the count.

The invention is therefore more functional and cost-effective.

The part of a device which remains in the surgical site allows the operator to see it even during the operation, and always evaluate the source and amount of haemorrhage (the absorbent part absorbs liquids highlighting the color of the fluid).

Surgical gauzes are strips of fabric, also stratified, which are known to be made of white cotton. Gauzes have an excellent liquid absorption capacity and are subjected to sterilization processes.

For the purposes of the present invention, the term device means gauze, swabs, sponge, used for all kinds of surgery, adapted to absorb body fluids.

During surgery, gauzes are used to pad and absorb blood and other body fluids in what is called the surgical site, or the portion of the patient's body in which the surgeon operates, such as the abdominal cavity.

Gauzes impregnated with blood and/or other body fluids basically take the same color of the tissues and/or fluids of the patient in the surgical site.

In other words, gauzes are “camouflaged” in the surgical site, thus becoming often difficult to identify, especially when the patient's bleeding is abundant.

This is the case for example with an operation in the abdomen of a patient.

Because of the change of color of the gauzes soaked with body fluids in the surgical site, the main risk in using them is to forget the gauze themselves inside the patient's body. The retention of gauzes in the human body can easily cause septicemia and infections and cause pain; the gauze retention may cause the patient's death, even some time after surgery.

The problem of retention of gauzes is one of the most felt worldwide.

Although no official incidence data is available, it is estimated that in Italy (data of the Ministry of Health) this phenomenon occurs once every 1000-3000 surgical procedures per year.

Various solutions have been proposed to overcome the problem.

A very common solution is to manually count the gauzes used each time by the surgeon or his team and check the numerical correspondence between the gauzes placed in the surgical site during the operation and used gauzes removed from the site after the operation (as mentioned above).

Any discrepancy indicates that one or more gauzes have been left in the patient's body and a verification is carried out.

Patent application U.S. 2008/0030303 describes gauzes provided with a visible number on the relevant surface to facilitate the manual count.

This methodology has been improved by providing the gauzes with labels readable by a scanner, such as bar codes.

Patent application EP-A-2399559 describes a method for counting the gauzes with a scanner manageable by the operator and connected to an electronic device which displays the counts related to each type of gauze used.

Patent application WO 2011/033540 describes a system comprising gauzes provided with RFID labels and and sensors able to detect such labels and transmit information about the presence of the corresponding gauze to a computer.

Despite the progress made, this solution has not proved to be 100% safe and alone is not sufficient.

Documents U.S. Pat. No. 7,465,847, U.S. Pat. No. 3,698,393, U.S. 20050109347, WO 2000/057783 and U.S. 2013/0035655, U.S. Pat. No. 4,244,369, describe different solutions all based on the following concept; each gauze is provided with an x-ray opaque insert.

After the operation and before concluding the final procedures of the operation, if the count indicates a numerical discrepancy between the gauzes introduced and those removed, the patient undergoes an x-ray to locate the radiopaque material each gauze is provided with.

These solutions are quite expensive, especially if compared with traditional gauzes and many hospitals in several countries could not adopt these gauzes for purely economic issues.

Another solution is to use chemicals that, applied to the gauze, enable the chromatic change thereof, i.e. change color upon contact with liquids, in particular blood or other organic liquids.

The aim is to maximize the chromatic contrast of the gauze with respect to the patient's tissues in the surgical site, in such a way as to visually bring out the gauze and facilitate the identification thereof by the operators.

For example, U.S. Pat. No. 3,731,685 describes a surgical gauze provided with a flap treated with these chemicals.

Patent application WO 2013/041976 by the Applicant describes an absorbent surgical swab whose surface is at least partly impregnated with dyes, which upon contact with the liquids of the surgical site are subject to colour change. When the change occurs, the swab is immediately evident even if the bleeding in the surgical site is abundant.

A similar solution is described in document CA 2022868.

A drawback of these solutions is the fact that in certain circumstances, the chemicals used to soak the gauze or swab can be released into the bloodstream of the surgical site and color the blood or tissues.

Another drawback is that once the gauze or swab have changed color, the surgeon is no longer able to easily distinguish the nature of the liquids present in the surgical site and absorbed by the same gauze or swab.

With the use of chemical reagents on the surface fabric of the gauze, having a dry white fabric that is optimal for distinguishing the origin of the haemorrhage once it comes into contact with the body fluids can become difficult.

Moreover, the use of reagents has limitations as regards the sterilization methods (for example, heat sterilization cannot be used if heat-sensitive molecules are involved).

For example, if the white gauze becomes blue for the effect of the chemicals contained therein, which react with the blood, the surgeon will not be able to easily distinguish a new loss of bile that adds up to the bleeding, because bile is green and this color may not be easily noticed on the blue gauze/swab.

On the other hand, it is desirable that the surgeon can easily and quickly identify which is the organ or tissue that at a given instant bleeds or loses fluids.

Other patents adapted to overcome the problem of retention of gauzes are the following:

WO 2009/000426 discloses a gauze detectable system with a chip connected to a device that emits a sound signal if a gauze remains within the surgical site at the end of the operation just before stitching up the patient.

WO 2005/066406 discloses a surface material already colored.

U.S. Pat. No. 3,731,685 discloses a surface chemical reagent in the tissue once-soaked.

U.S. Pat. No. 3,888,248 refers to the post-operative scope.

U.S. Pat. No. 4,917,694 discloses a radiopaque detectable material.

U.S. 2012/0259302 includes detectable items.

U.S. 2012/0079590 discloses a device that allows an order in the arrangement for counting the gauzes.

WO 00/57783 discloses a smart detectable system that can track down the device by means of electromagnetic field variations or rx.

WO 02/45763 discloses a surface fabric with built-in antimicrobial features.

U.S. Pat. No. 4,327,731 discloses a layer detectable with chemical reagents which can include a tear-off system which can be associated to the reactive layer (the technical field of this invention is not provided in the operating room).

U.S. Pat. No. 4,910,803 discloses a first layer of liquid repellent fabric, moreover it discloses gloves and masks used in the operating room.

U.S. Pat. No. 795,491 discloses a method for counting the gauzes with detectable aids.

U.S. Pat. No. 7,297,834 discloses a sponge with a numerical support on the outer surface.

CA 1114354 discloses a first layer which is blood repellent.

CA 2022868 discloses an already colored gauze.

U.S. Pat. No. 5,931,824 discloses the identification of gauzes with a surgical procedure using a detectable signal (scanning beam).

U.S. Pat. No. 5,045,080 and U.S. Pat. No. 3,965,907 disclose rx markers.

### BRIEF SUMMARY OF THE INVENTION

The object of the present invention is to provide a biomedical device for surgery which overcomes the drawbacks of the traditional solutions.

According to the invention, said and further objects are achieved by a surgical gauze as described in claim 1.

The present invention allows a constant area of landmark surgery used operationally by the operator, so as to facilitate the operation in terms of viewing of the surgical site; for example, in vascular surgery, placing a white landmark surgery gauze below the artery to be sutured makes the view of the surgical site immediate and delimited, and thus the operator will probably spend less time in suturing such an artery.

The claimed device advantageously allows rapid identification of the nature of the fluids present in the site itself allowing the control of the haemorrhage/loss of body fluids; being visible, the device allows the operators to prevent the risk of retention of gauzes at the end of the operation.

Said device can also be used to enhance the safety of the methodology of counting the gauzes since the count itself is double.

Being a consumer device, it reflects the needs of safety and saving and poses no limits to the sterilization methods.

### BRIEF DESCRIPTION OF THE SEVERAL VIEWS OF THE DRAWINGS

Further features and advantages of the invention will appear more clearly from the following detailed description of a preferred but non exclusive embodiment, shown by way of a non limiting example with the aid of the accompanying drawings, in which:

FIG. 1 shows a section (which may also be considered a lateral view) where the assembly (medical device) configuration is shown;

FIG. 2 shows an axonometric view of the assembly (medical device);

FIG. 3 shows the removal of a first layer;

FIG. 4 shows the first layer removed;

FIG. 5 shows the device after the removal of the surface layer;

FIG. 6 shows a schematized vertical sectional view of a gauze according to a second embodiment of the invention, in rest position;

FIG. 7 shows a view similar to that in FIG. 6 of the same gauze in working position;

FIG. 8 shows a top plan view of a gauze according to a third embodiment in which the second layer is in strips, in working position;

FIG. 9 shows a top plan view of the gauze in FIG. 8 with a closing and counting band;

FIG. 10 shows a schematized vertical sectional view of a gauze according to a fourth embodiment of the invention, in rest position;

FIG. 11 shows a schematized vertical sectional view of a gauze according to a fifth embodiment of the invention, in rest position;

FIG. 12 shows a top plan view of a gauze according to a sixth embodiment of the invention, as a “block notes”.

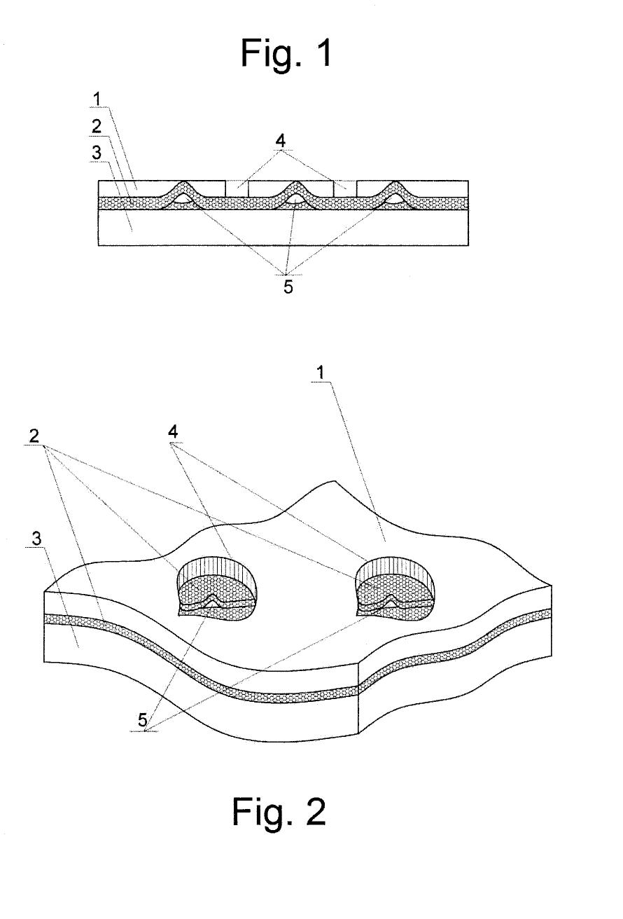

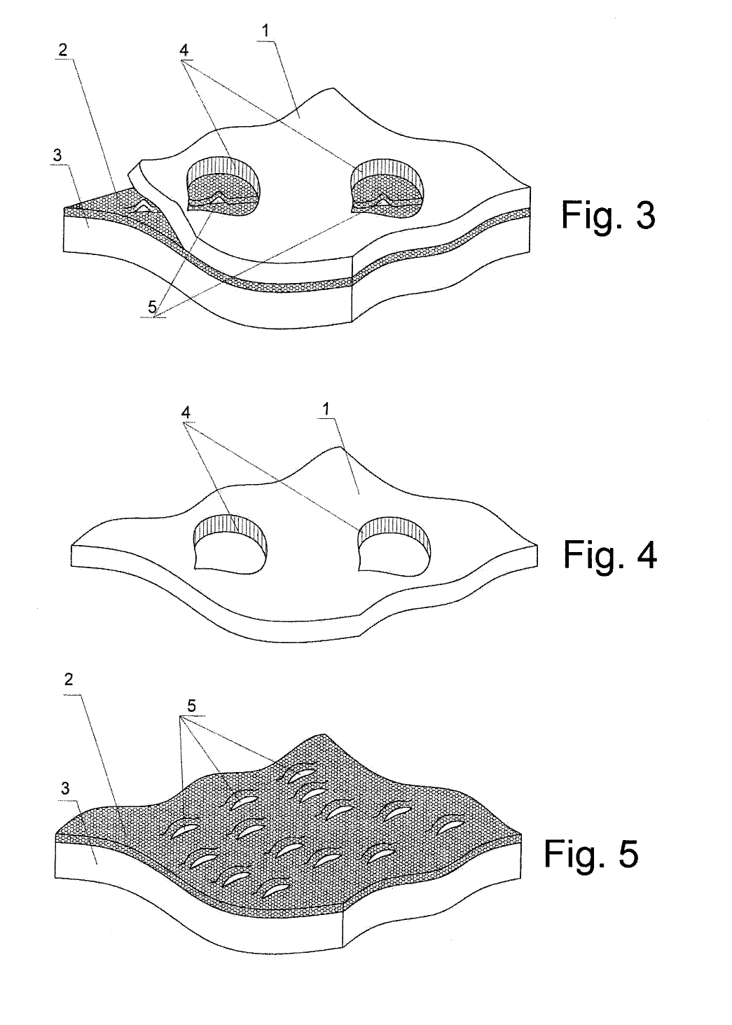

### DETAILED DESCRIPTION OF THE INVENTION

A surgical gauze includes a first layer 1, a second 2 and a third layer 3 overlapping one another, in direct contact.

The first layer 1 is made of a material absorbing the liquids in a surgical site and the second layer is made of a material not absorbing such liquids, i.e. a substantially waterproof material, such that in contact with the body fluids, it keeps its light or dark color, acting as landmark surgery for the operator, while the fabric of the third layer 3 absorbs the fluids even under the waterproof layer; the shape/morphology of the waterproof layer is such as to give the operator a defined and not discontinuous (optimum operationally) chromatic view and at the same time make the liquids flow in the underlying absorbent fabric; the waterproof layer 2 and the underlying absorbent layer 3 are irreversibly attached.

The first layer 1 is provided with one or more windows 4, or in general openings that allow the surgeon to see corresponding portions of the second layer 2, although substantially covered by the first layer 1.

Therefore, even if the first layer 1 becomes impregnated with blood or other fluids, taking the color thereof and actually camouflaging in the surgical site, there is always a visual window 4 for the operator (opening/openings on the first absorbent layer 1 such as to let the detector layer thereunder be visible); when the first layer 1 is removed (at the discretion of the operator), likely if the haemorrhage is controlled, the other function of the device that is being landmark surgery for the operation becomes fundamental.

The second layer 2 ensures the visibility of the gauze because it does not change color, and acts as a contrast element with respect to liquids and tissues present in the surgical site.

The gauze thus conceived has an operative orientation adapted to facilitate the function of the surgeon; in fact, the advantage that the first layer 1 is removable firstly allows controlling the hemorrhage without altering the chromatic aspects of the fluids which impregnate the fabric, giving a visual perception of the origin and the amount of fluid exiting from human tissues in the surgical site, and secondly, when the hemorrhage is controlled, at the discretion of the operator, it allows to be easily removed, thus becoming the landmark surgery device to facilitate the operators work, ensuring a clear distinction between the tissues on which he has to operate and the gauzes, while maintaining the hemostatic function of the device.

After removing the surface layer, the fluids can still flow from the waterproof layer to the absorbent layer underneath, while ensuring the hemostasis through windows 5 adapted to let the liquids be absorbed by the underlying absorbent fabric and in any case to give a chromatic continuity of the device to the operator a to facilitate the surgeon's operation.

In substance, the fluids are absorbed in part by the first layer 1, then flowing through the fabric and through windows 4 of the first (absorbent) layer 1 and windows 5 of the second (waterproof) layer 2, they are absorbed by the third layer 3 until saturation.

A further advantage provided by the gauze according to the present invention is given by the fact that, thanks to the chromatic contrast provided by the second layer 2 with respect to the liquids of the surgical site, the surgeon can identify at any time the nature of these liquids.

The fact that the device is made in this way offers a landmark surgery, partial before use and total after the removal of the surface layer, without neglecting the distinction of color given by the absorption of fluids on absorbent fabrics, which however do not change in color, allowing the surgeon to get oriented from the operational point of view (which is missing with fabrics that irreversibly change color and do not allow the operator to understand which fluid is leaking and in what quantity).

For these reasons, preferably, the second layer 2 (non-absorbent and waterproof) can be of a light color, preferably white.

Alternatively, the second layer 2 (non-absorbent and waterproof) can be of a dark color, preferably blue.

The second layer 2 may also be conceived by combining the two tones and in different shapes adapted to give a landmark surgery area for the various types of surgery.

A device may also be conceived, still according to the present invention, in which there is no orientation but also made symmetrically with multiple elements of the type with “first layer” (on one side and on the other of the whole of gauze), multiple waterproof layers with multiple absorbent layers inside.

In this case, the device is symmetrical in its construction and the counting of the gauzes will thus be reformulated, i.e. a proportion of 2:1 between fabrics removed and fabric with waterproof surface above and below with absorbent fabric inside.

A variant of the fabrics in multiple layers (with two waterproof layers) allows simultaneously removing both removable fabrics in order to maintain the proportion of 1:1 in the counting step.

Therefore, the gauze according to the present invention also is a valid aid for the surgeon since it facilitates the identification of the organs on which to intervene.

In the preferred embodiment, the first layer 1 and the second layer 2 are removably joined, for example coupled via a yarn interlocking, so as to be separable by the surgeon with an easy extraction maneuvre, manually or with the aid of pliers.

Preferably, the absorbent layers may be made of hydrophilic fabric, such as cotton.

Preferably, also the second layer 2 may be white and/or blue, colors adapted to be well distinguishable within the surgical site.

In one embodiment, the absorbent fabrics may contain agents that promote the hemostasis, such as potash alum or hygroscopic molecules adapted to maximize the absorbent function, potentially reducing the number of devices for the operation and increasing the economic advantage.

The method for introducing agents which promote hemostasis and/or hygroscopic molecules without the danger of releasing substances within the surgical site is by mordanting.

Mordanting with potash alum is an example that can be, alone or combined with hemostatic and/or hygroscopic agents, used to improve the functionality of the device.

This solution also allows maximizing absorption.

Mordanting is little expensive and routine in the preparation of a fabric, which ensures that functional molecules can be irreversibly retained within a fabric and therefore not be released.

In the preferred embodiment, the second layer 2 is a fabric, or a non woven fabric, of synthetic material with the following features:

thermoplasticity (to make the shape of the fabric suitable for the flow of fluids);

static nature (the fabric should not be elastic). To this end, the construction of the fabric is achieved by using orthogonal frames, which make a weave with perpendicular yarns;

waterproof (essential for the function of landmark surgery).

For example, a particularly suitable material to make the second layer is cationic polyester, which is hot and sterilizable, has the advantage of being inherently waterproof without the addition of any treatment (and therefore economically more advantageous), and is compatible with a consumer device from the economic point of view.

Alternatively, polypropylene, polyamide, polyethylene terephthalate (PET), carbon, silk, acrylic are equally usable.

A potentially interesting aspect inherent in polyester fibers, PET, carbon relates to the reuse by recovery of the second layer with relative washing and application on a new device, ensuring the initial features thereof as unchanged.

The term “layer” in the present patent application is intended to identify a thickness, even if formed by one or more flaps of cotton, one or more flaps of fabric or nonwoven fabric wrapped or folded on one another.

It will be apparent to the man skilled in the art that each layer of the gauze can in turn contain multiple layers of the same material or of different materials, provided that together they have the technical features described.

With patent application WO 2013/041976, in the name of the Applicant, a solution related to chemical reagents has been overcome with this patent application, eliminating the occurrence of any kind of chemical or other reagent, allowing maximizing the functionality of the gauze (haemorrhage control and landmark surgery function) with a simple production process, not harmful to the health and cost-effective, bypassing the problems that could be caused by the reagent and/or mixtures in the gauze that were at risk of release into the surgical site; moreover, standardizing a chemical reaction within the surgical site may be complex and/or dangerous, in addition to expensive.

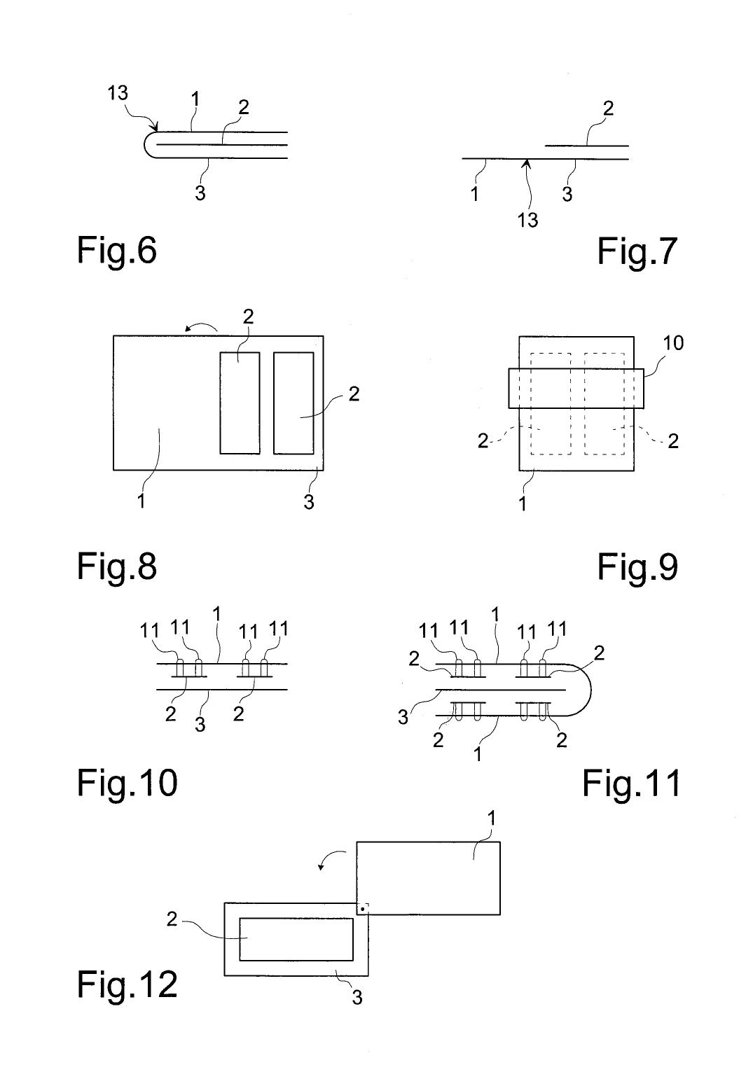

FIGS. 6-9 show embodiments of the invention in which the surgical gauze provides the first 1 and the third layer 3 joined “book-like”. Compared to the first embodiment, when the gauze is placed in the surgical site (working position) the first layer 1 is not removed but it simply opens “book-like”, leaving the second layer visible (FIG. 7). with the same effect as described above.

In substance, the first 1 and the third layer 3 form a single absorbent layer 13.

The function of landmark surgery can be maintained using a removable ring band 10 which prior to placing the gauze in the surgical site (rest position), allows keeping the gauze closed (FIG. 9).

In FIGS. 8-11 it can be seen that the second waterproof layer 2 is in bands.

FIG. 10 shows (absorbable or not absorbable) stitches 11 which allow keeping the first layer 1 associated with the second layer 2 before placing the gauze in the surgical site (rest position), said stitches 11 allowing easy extraction of the first layer 1, i.e. the removal thereof for the landmark surgery function.

In the embodiment of FIG. 10, a central hole may be provided in the first layer 1 to facilitate the removal thereof.

In the embodiment shown in FIG. 11, a second layer 2 is also present under the third layer 3, and the first layer 1 covers said second layers 2 both above and below. As in the embodiment in FIG. 10, the first layer 1 is associated to the second layer 2 by stitches 11.

The embodiments of FIGS. 10 and 11 define a “rack” system, in which stitches 11 are partly or totally elastomeric.

Alternatively, the first layer 1 can be associated to the underlying layers 2, 3 in a single point, thus being easily removable by tearing.

Still alternatively, the first layer 1 may be as a “cuff”, thus enclosing said second layer 2 and third layer 3 therein, said “cuff” being easily openable for easy removal from the surgical site with a landmark surgery function.

FIG. 12 shows a “block notes-like” surgical gauze, i.e. with the first layer 1 attached to the third layer 3 at a point, rotatable about said point so as to cover or not the second layer 2, and removable by tearing for the landmark surgery function.

Stitches 11 may be of Solvron, a material that melts in contact with the blood, thus allowing the removal of the first layer 1.

The coupling between the first layer 1 and the other two layers 2, 3 may be done with CO2 laser, heat-setting or by ultrasounds.

It is noted that the landmark surgery layer may be made in bands or strips or chess, or in any pattern such as to maximize the functions of absorption and of landmark surgery of the gauze.

1. A surgical gauze, wherein it comprises a first layer of absorbent fabric, a second layer of waterproof fabric and a third layer of absorbent fabric,
said second layer being interposed between said first layer and third layer,
said first absorbent layer which is on top of the second waterproof layer being removable from a surgical site to combine the haemostatic, landmark surgery and counting function,
said first layer being provided with one or more openings to always let said second layer be visible before and also after placing the gauze in the surgical site,
said second layer having a morphological shape which provides for windows that allow the passage of fluids thereunder in order to be absorbed by said underlying third fabric, thus maintaining an optimal chromatic continuity to maximize the vision of the surgical site,
said first and third absorbent layers being made of hydrophilic fabric,
said second waterproof layer being a fabric, or a nonwoven fabric, the second waterproof layer and the underlying third absorbent layer being irreversibly joined,
the second waterproof layer being immediately distinguishable in the surgical site since it is in chromatic contrast with body fluids/tissues absorbed by said first layer, between light and dark shades.

2. The gauze according to claim 1, wherein the first and the second layer are joined together with stitches or are ultrasonically welded in one or more points.

3. The gauze according to claim 1, wherein the color of the polyester fabric of the second layer is given by basic dyes.

4. The gauze according to claim 1, wherein each of said layers is a multilayer.

5. A surgical gauze, wherein
it comprises a first layer of absorbent fabric, a second layer of waterproof fabric and a third layer of absorbent fabric,
said second layer being interposed between said first layer and third layer.
said first absorbent layer which is on top of the second waterproof layer being joined “book-like” to said third layer so that when the gauze is placed in the surgical site, the first layer is not is removed but it simply opens “book-like”, leaving the second layer visible, said first layer and third layer forming a single absorbent layer,
said first layer being provided with one or more openings to always let said second layer be visible before and also after placing the gauze in the surgical site,
said second layer having a morphological shape which provides for windows that allow the passage of fluids thereunder in order to be absorbed by said underlying third fabric, thus maintaining an optimal chromatic continuity to maximize the vision of the surgical site,
said first and third absorbent layers being made of hydrophilic fabric,
said second waterproof layer being a fabric, or a non-woven fabric, either synthetic or natural,
the second waterproof layer and the underlying third absorbent layer being irreversibly joined,
the second waterproof layer being immediately distinguishable in the surgical site since it is in chromatic contrast with body fluids/tissues absorbed by said first layer, between light and dark shades.

6. The gauze according to claim 5, wherein it comprises a removable ring band serving as landmark surgery, which before placing the gauze in the surgical site allows keeping the gauze closed.

7. The gauze according to claim 1, wherein the second waterproof layer is in bands, said windows corresponding to spaces between two adjacent bands.

8. The gauze according to claim 7, wherein it is provided with absorbable or not absorbable stitches which allow keeping the first layer associated with the second layer before placing the gauze in the surgical site, said stitches allowing easy extraction of the first layer.

9. The gauze according to claim 8, wherein it is provided, with a central hole in the first layer to facilitate the removal thereof.

10. The gauze according to claim 1 wherein a second absorbent layer is also present under the third layer,
the first layer covering said second layer both above and below.
